(19) United States
(12) Patent Application Publication
(10) Pub. No.: US 2026/0327164 A1
(43) Pub. Date: Oct. 22, 2026

(54) Method and a System for Producing One or More Olefins

(71) Applicant: Linde GmbH, Pullach (DE)

(72) Inventors: Torben Höfel, Munchen (DE); Gunther Kracker, Weyarn (DE); Gunther Schmidt, Pullach (DE)

(21) Appl. No.: 19/113,179

(22) PCT Filed: Sep. 14, 2023
(86) PCT No.: PCT/EP2023/075286

§ 371 Date: Mar. 19, 2025

(30) Foreign Application Priority Data
Sep. 19, 2022 (EP) 22020449.9

(51) Int. Cl. C07C 5/327; B01D 53/04; B01D 53/14; B01D 53/22

(52) U.S. Cl. CPC C07C 5/327; B01D 53/04; B01D 53/1475; B01D 53/229; B01D 2256/16; B01D 2256/245; B01D 2257/504; B01D 2258/0283

(57) ABSTRACT

A process for producing an olefin includes reacting a paraffin to obtain a product mixture in a reaction step. The reaction step is carried out using heat of combustion. The product mixture contains the olefin, hydrogen and methane. A tail gas is formed using the product mixture in (a) separation step(s). The tail gas is substantially free of the olefin and contains the hydrogen and the methane from the product mixture. The tail gas is permanently discharged from the process, wherein the heat of combustion is provided using a fuel gas. A mole ratio of hydrogen to methane in the tail gas is at least as high as in the product mixture and in the fuel gas is lower than in the tail gas. A flue gas containing carbon dioxide is formed during the provision of the heat of combustion; the flue gas is subjected to carbon dioxide capture.

### CROSS-REFERENCE TO RELATED APPLICATIONS

This application is the national phase of, and claims priority to, International Application No. PCT/EP2023/075286, filed 14 Sep. 2023, which claims priority to European Patent Application No. EP22020449.9, filed 19 Sep. 2022.

### FIELD OF THE INVENTION

The invention relates to a process and to a system for producing one or more olefins.

### BACKGROUND

Processes and systems for the production of olefins (alkenes), in particular from paraffins (alkanes), are known. Among many others, reference is made to the articles “Ethylene” and “Propylene” in Ullmann's Encyclopedia of Industrial Chemistry, online publications dated Apr. 15, 2009 and Sep. 16, 2013.

Processes and systems for producing olefins can be highly selective, meaning that, for example, greater than 75 mol. % of the carbon from the feed used can end up in the desired product, with little carbon in by-products such as methane. Corresponding processes can be endothermic and non-oxidative, i.e., the energy supply can be via a separate energy source. Electrical current can be used or a fuel can be burned for heating, although different variants are known for each. The present invention is not limited to a specific variant.

In addition to direct resistive heating of the catalyst itself or of a carrier to which it is applied, for example by means of a wash coating, resistive heating elements can also be used for heating with electrical current. It is also possible to electrically superheat the process gas, which then flows through a catalyst bed at an increased temperature (if a catalyst is used). In this case, an adiabatic reaction is implemented. Several corresponding superheating steps and subsequent adiabatic catalyst beds in series can also be provided.

In addition to direct heating, in which the heating medium, for example a heating element, a heating sleeve or a resistively heated catalyst carrier, is in material contact with the process gas or in material contact with reaction tubes through which the process gas flows, indirect electrical heating can also be used, as explained, among other places, in WO 2020/002326 A1—and also already known from earlier publications.

Such indirect electrical heating can be carried out using electrical radiant heating elements which are suitable for heating to the high temperatures required for the reactions mentioned, such heating elements being arranged such that they are not in direct contact with the reaction tubes. The heat transfer occurs predominantly or exclusively in the form of radiant heat.

Heating by means of combustion can take place in a furnace or heat exchanger with a radiant zone, wherein burners in different numbers, positions and arrangements can be used in a combustion chamber, which can be operated with a wide variety of fuel gases and with or without the addition of other combustible media, and with or without heating of the fuel gas or of combustion air used for combustion, or other oxygen-containing gas mixtures. Here, too, intermittent heating of a catalyst bed by means of oxidation or the use of a fluidized bed reactor with separate reaction chamber and combustion chamber is possible. Heat recovery, for example in a convection zone, is also possible. This heat recovery can be used, for example, for preheating raw materials, for steam generation, for steam heating, for air preheating and the like.

Paraffins or other carbon-containing feedstocks used for the production of olefins can also be suitable for the production of hydrogen during the reaction. This applies, for example, to paraffinic compounds with two to four carbon atoms, but to a certain extent also to long-chain hydrocarbons such as methanol or ethanol.

A product mixture of such processes contains comparatively little methane and comparatively much hydrogen in the so-called C1minus fraction. Typical examples are 80 mol. % hydrogen and 20 mol. % methane. The C1minus fraction comprises in particular compounds with one carbon atom, including possibly carbon monoxide and carbon dioxide, the aforementioned methane, and lighter compounds such as the aforementioned hydrogen.

Corresponding values apply, for example, to so-called gas crackers, i.e., cracking furnaces designed for gaseous feedstocks in steam cracking processes, and propane dehydrogenations.

Basically, the processes mentioned produce highly selective olefins and a C1minus “energy stream” (named because of the typical use for combustion, i.e., energy use) with low so-called carbon dioxide intensity. In the case of propane dehydrogenation, this stream may also contain, for example, small amounts of hydrocarbons with two carbon atoms. In the following, this stream is referred to as tail gas. It typically comprises all or a substantial part, in particular greater than 90% or 95% of all components of a corresponding type from the product mixture of a corresponding process.

The term carbon dioxide intensity correlates with the amount of carbon dioxide produced by the combustion of a gas or gas mixture or other fuel. For example, a hydrogen-free methane stream (e.g., natural gas, with the simplified assumption of a content of 100% methane) has a carbon dioxide intensity of approximately 0.2 tons per hour of carbon dioxide per megawatt, based on the lower calorific value. A tail gas of the type described, with, for example, 80 mol. % hydrogen and 20 mol. % methane, on the other hand, only has a carbon dioxide intensity of approximately 0.09 tons per hour of carbon dioxide per megawatt, based on the lower calorific value.

Typically, corresponding tail gases are intended directly for underfiring in the process or system for the production of olefins. If, to put it simply, one assumes an identical energy consumption of any process or system of this type (in gigajoules per ton of value products), the carbon dioxide intensity of the process underfired with hydrogen-rich tail gas is only 45% compared to a process underfired with methane. This can be a decisive advantage. As shown above, highly selective processes for the production of olefins typically have low carbon dioxide production, which can be, for example, 0.4 tons of carbon dioxide per ton of value products or even less.

However, if the aim is to decarbonize direct emissions (“Scope 1” emissions) as much as possible (e.g., by 95%), difficulties may arise in the corresponding processes. The invention therefore has the object of providing advantageous solutions for such cases.

### SUMMARY

As already indicated above, it can be seen that the processes for producing olefins described above already have extremely low specific direct (“Scope 1”) carbon dioxide emissions. However, these emissions are particularly difficult to reduce completely.

Full electrification of all energy supplies, including the reactors used, is not yet commercially available, or may prove to be costly. Underfiring with a very hydrogen-rich combustion medium (approx. 99 to 100 mol. % hydrogen) would in principle be suitable for reducing such emissions. The first option for this is hydrogen from the aforementioned tail gas, from which the methane content would have to be captured. However, the hydrogen from the tail gas is usually not sufficient to cover the energy demand, even if the process is highly energy-optimized. In a typical gas cracker, for example, hydrogen is sufficient for about 50% of the energy supply.

It would therefore be plausible to first try to optimize the energy use of the process, in particular with the partial aid of electrification. Finally, an additional supply of hydrogen would be necessary. In particular, so-called “green” hydrogen from water electrolysis powered by renewably generated current comes into consideration. However, this option can be very costly and energy intensive. On the other hand, “blue” hydrogen, e.g., from methane reforming with separation of carbon dioxide from the flue gas (carbon capture), could also be used. However, this variant is also associated with significant heat losses of the methane used and is costly.

Instead of an a priori reduction of the carbon dioxide content in the flue gas, emissions can also be reduced by separating carbon dioxide in the flue gas of the combustion processes or equipment used in the olefin production processes (cracking furnaces, fired heaters, boilers, regenerators for catalysts, etc.). In a corresponding flue gas, however, the carbon dioxide is present at very low pressure/partial pressure, which is further reduced by the high proportion of hydrogen combustion.

The invention now solves these problems at least partially by deliberately refraining from burning the hydrogen-rich tail gas, instead burning methane or a hydrocarbon-rich fuel, and releasing the tail gas or hydrogen therefrom—in particular, to the system boundary. This seems counterintuitive at first glance, since avoiding all carbon dioxide formation initially seems desirable, which is precisely what the invention does not propose.

The solution proposed here is therefore diametrically opposed to prior art solutions in which hydrogen-rich fuel gas streams are generated.

For example, WO 2023/049570 A1 proposes the production of a hydrogen-rich fuel gas stream by reforming a hydrocarbon-containing feed and subjecting the resulting process gas to a water/gas shift and carbon dioxide separation. According to the embodiments cited there, the remaining hydrogen-rich stream can advantageously be burned to provide thermal energy.

EP 3,249,027 A1 proposes a process for producing olefins by steam cracking using one or more tubular reactors. The tubular reactor or at least one of the multiple tubular reactors is heated using both heat of combustion generated by burning at least one fuel and electric heat generated by means of electrical energy. However, it is not proposed to discharge a hydrogen-rich stream from the process rather than burning it.

The invention typically comprises the reaction of a paraffinic feed which typically contains predominantly or exclusively hydrocarbons having two to four carbon atoms. For example, ethane, propane or liquefied petroleum gas (LPG) can be used. In order to obtain olefins, in particular ethylene and propylene, a product mixture is formed which contains these olefins. The product gas also contains at least methane and hydrogen, with a mole ratio of hydrogen to methane of greater than 0.4, in particular greater than 1, and further in particular greater than 2 or greater than 4. The energy demand of the process is at least partially covered by underfiring a hydrocarbon or hydrogen or a mixture of these.

Within the scope of the invention, a product fraction is then discharged from the process and/or released at the system boundary; this substantially comprises compounds with one carbon atom and/or lighter compounds. This can be a tail gas or a fraction that is formed from it. A mole ratio of hydrogen to methane in this tail gas is at least as high or higher than the mole ratio of hydrogen to methane in the product mixture. An energy stream, i.e., a fuel gas, is supplied to the process externally, i.e., from outside the system boundary, and serves to supplement the underfiring. The mole ratio of hydrogen to methane in this energy stream is lower than that of the tail gas and/or a fraction formed from it. During underfiring, a flue gas is formed, at least part of which is supplied to a carbon capture process.

The term “substantially” is to be understood here in particular to mean that in a claimed composition, a material stream, etc., in addition to the components indicated as mandatory, further components may be present, provided that the substantial characteristics of the claimed composition are not significantly changed by these further components. For example, a product fraction comprising “substantially compounds with one carbon atom and/or lighter compounds” contains hydrocarbons with two or more carbon atoms, in particular olefins, only to a very small extent, so that their release at the system boundary results in only very small losses of value products. In particular, this may be less than 1, 0.1, or 0.01% of such higher hydrocarbons. The percentages can refer to molar, quantitative or volume fractions.

Overall, the invention proposes a process for producing one or more olefins, wherein one or more paraffins are reacted to obtain a product mixture in one or more reaction steps, wherein the reaction step(s) are carried out using heat of combustion, wherein the product mixture contains the one or more olefins, hydrogen and methane, wherein a tail gas is formed using the product mixture or a part thereof in one or more separation steps, wherein the tail gas is substantially free of the one or more olefins and contains the hydrogen and the methane from the product mixture or a part thereof, wherein the tail gas or a part thereof is permanently discharged from the process, wherein the heat of combustion is provided using a fuel gas, wherein a mole ratio of hydrogen to methane in the tail gas is at least as high as in the product mixture, and in the fuel gas is lower than in the tail gas, wherein a flue gas containing carbon dioxide is formed during the provision of the heat of combustion, and wherein the flue gas or a part thereof is subjected to carbon dioxide capture.

The advantages of the process proposed here are in particular that the separation of carbon dioxide from the flue gas is simplified due to the higher partial pressure. The previously explained difficulties of a complete decarbonization using a variant involving electrification and pure hydrogen combustion options currently appear insurmountable or unrealistic. Accordingly, in the scope of the invention, carbon dioxide removal from flue gas is optimized. The invention provides surprising synergy effects.

The invention comprises permanently discharging the tail gas or a part thereof from the process. The term “permanently discharged from the process” means that the tail gas or its discharged part, which in particular has the same hydrogen content as the tail gas, is not combusted in the process to generate heat- or only a small part of it is, for example less than 10%, 5% or 1%. Instead, a low-hydrogen or (substantially) hydrogen-free fuel gas is burned. A tail gas or a corresponding part permanently discharged from the process is released in particular at a system boundary of a system used in the process and is not returned to the process itself or a corresponding system. In this way, corresponding hydrogen does not “dilute” the flue gas and thus cannot make carbon dioxide separation more difficult.

For example, in embodiments of the invention, the fuel gas or a part thereof can be taken from a gas supply line (pipeline) and the tail gas, or the part thereof permanently discharged from the process can be fed back into the gas supply line. In this way, the process proposed according to embodiments of the invention can capture a greater amount of carbon dioxide than would be possible with complete internal utilization of the hydrogen-enriched tail gas. A system according to an embodiment of the invention thus serves for additional carbon dioxide removal. At the same time, in corresponding embodiments of the invention, an export stream with lower carbon dioxide intensity is available, which can be provided for use elsewhere. In addition to removal and feed-back from or into the same pipeline, removal may also be provided from a source that is different from a sink into which the feed-back takes place.

In the illustrated example, a device such as a steam cracker can be used to partially decarbonize a gas pipeline. The raw material with a carbon dioxide intensity of 0.2 tons per hour of carbon dioxide per megawatt, based on the lower calorific value, such as methane-rich natural gas, is partially replaced by tail gas with a carbon dioxide intensity of, for example, 0.09 tons per hour of carbon dioxide per megawatt, based on the lower calorific value. Advantages can be achieved here (e.g., through certification processes), since in olefin production the carbon dioxide is actually not released into the atmosphere, thanks to carbon dioxide capture.

In embodiments of the invention, the tail gas or a part thereof may be subjected to one or more tail gas separation steps to obtain a first tail gas fraction which is enriched in hydrogen and depleted in methane compared to the tail gas, and a second tail gas fraction which is depleted in hydrogen and enriched in methane compared to the tail gas. In this way, in particular a pure hydrogen product can be provided in the form of the first tail gas fraction.

By (at least partially) separating methane and hydrogen in appropriate designs, an export stream with even lower carbon dioxide intensity can be generated, down to a carbon dioxide intensity of zero (hydrogen). The exported hydrogen is therefore associated with very low direct carbon dioxide emissions (“blue” hydrogen), provided that, for example, greater than 60%, or in particular greater than 95%, of the carbon dioxide is captured from the flue gases.

In any case, the first tail gas fraction or a part thereof can be permanently discharged from the process, but it is also possible to use the first tail gas fraction or a part thereof separately from the fuel gas to provide further heat of combustion. Due to the preferably pure hydrogen combustion, no carbon dioxide separation is carried out in the resulting exhaust gas. The second tail gas fraction or a part thereof, however, can, due to the corresponding methane content, be used for the “regular” combustion, downstream of which the carbon dioxide capture takes place.

In embodiments of the invention, the one or more tail gas separation steps may comprise one or more membrane separation steps and/or one or more adsorptive separation steps. These can take place directly in the separation part or in a separate unit that processes the tail gas. The separation may comprise compression and cryogenic separation, as well as (vacuum) pressure swing adsorption, membrane processes or a combination of the above-mentioned processes, as are generally known to a person skilled in the art.

As already mentioned, in embodiments of the invention, greater than 60% or 95% of the carbon dioxide contained in the flue gas or the part thereof subjected to carbon dioxide capture can be captured during carbon dioxide capture. This results in the advantages mentioned above.

In embodiments of the invention, carbon dioxide capture can be carried out using one or more absorption liquids, as is also known in principle. For example, amine and caustic washes can be used alone or in any combination. Alternatively, simple compression and condensation can be provided (in the case of the oxyfuel furnaces explained below).

In embodiments of the invention, the fuel gas or a part thereof may be combusted using an oxygen-enriched oxidizer gas. The oxidizing agent in such ovens can therefore be enriched with oxygen or even be (almost) completely oxygen (so-called oxyfuel furnaces). In this case, carbon capture from these furnaces becomes particularly easy since the flue gas consists almost entirely of hydrogen and carbon dioxide.

If possible, the reaction step(s) may also be carried out using electrically supplied heat. This reduces the need to import fossil fuels.

In embodiments of the invention, the fuel gas may be a methane-rich gas or gas mixture. For example, it can be fossil methane, e.g., natural gas, where the carbon dioxide formed can be almost completely (e.g., 95%) removed from the flue gas, as described above. This emission reduction can be credited to the olefin product and/or the hydrogen-rich export stream.

Supplementation with biogas may also be provided. Since the carbon from the biogas is almost completely removed from the flue gas in the carbon capture process, this produces “negative” direct carbon dioxide emissions (“bio-carbon dioxide” is captured). These negative emissions can be credited to the olefin product and/or the hydrogen-rich export stream. A feed can also be of fossil origin, or from a circular or biogenic source. The prerequisite is that this feedstock generates a high hydrogen/methane ratio during the reaction.

A hydrogen-rich stream formed can have a low pressure if it is used directly for underfiring. Alternatively, it can have a high pressure if this stream is fed into any hydrogen value chain and/or pipeline. Accordingly, specifications can vary considerably, from typical tail gas to 99.9999 mol. % hydrogen.

A system for producing one or more olefins is designed to convert one or more paraffins to obtain a product mixture in one or more reaction steps, to carry out the reaction step(s) using heat of combustion, to form the product mixture in such a way that it contains the one or more olefins, hydrogen and methane, to form a tail gas using the product mixture or a part thereof in one or more separation steps, to form the tail gas in such a way that it is substantially free of the one or more olefins and contains the hydrogen and methane from the product mixture or a part thereof, to permanently discharge the tail gas or a part thereof from the system, and to provide the heat of combustion using a fuel gas, wherein a mole ratio of hydrogen to methane in the tail gas is at least as high as in the product mixture and in the fuel gas is lower than in the tail gas, wherein a flue gas containing carbon dioxide is formed during the provision of the heat of combustion, and wherein the flue gas or a part thereof is subjected to carbon dioxide capture.

For further features and advantages of a corresponding system and embodiments thereof, express reference is made to the above explanations concerning the process proposed according to the invention and its embodiments, since these apply to this in the same way.

The same applies to a system which is designed to carry out a process according to any embodiment of the invention.

Before describing embodiments of the invention with reference to the accompanying drawings, some further aspects of the invention and of embodiments not according to the invention will be briefly explained, which can be used in any combination with the embodiments explained above and below.

In embodiments of the invention, a separation of methane and hydrogen in a gas cracker can also serve the purpose of exporting methane, with the aim of, among other things, direct export to a pipeline, hydrogen production, methane pyrolysis, synthesis gas production, other processes for producing value products or use in a power system.

Separating methane and hydrogen in a gas cracker can make sense even if neither hydrogen nor methane is exported. The separation enables different processes to be underfired with either hydrogen or methane. This means that only the carbon dioxide from the methane-underfired processes needs to be captured.

The separation of hydrogen and methane can also be carried out in combination with the use of appropriately tailored furnace designs; and various “hybrid” system designs can be envisaged according to embodiments. Examples include hydrogen furnaces with hydrogen combustion combined with methane furnaces (and optional carbon dioxide capture), hydrogen furnaces with hydrogen combustion combined with methane oxyfuel furnaces (and optional carbon dioxide capture), and hydrogen furnaces with hydrogen combustion combined with electric furnaces and methane export to a value chain.

Biogas can be imported even if no carbon dioxide is captured. In this way, energy export with low carbon dioxide intensity would also be possible (but not a negative carbon dioxide balance).

### BRIEF DESCRIPTION OF THE DRAWINGS

Embodiments of the invention are described below purely by way of example with reference to the accompanying drawings, wherein:

FIG. 1 illustrates a system according to an embodiment of the invention, and

FIG. 2 illustrates a system according to an embodiment of the invention.

### WRITTEN DESCRIPTION

The embodiments described below are described solely for the purpose of assisting the reader in understanding the features claimed and previously discussed. They are merely representative examples and are not intended to be exhaustive and/or limiting with regard to the features of the invention. It is to be understood that the advantages, embodiments, examples, functions, features, structures and/or other aspects described above and below are not to be considered as limitations on the scope of the invention as defined in the claims or as limitations on equivalents to the claims, and that other embodiments may be utilized and changes may be made without departing from the scope of the invention as claimed.

Different embodiments of the invention may comprise, have, consist of, or consist substantially of other useful combinations of the described elements, components, features, parts, steps, means, etc., even if such combinations are not specifically described herein. Furthermore, the disclosure may cover other inventions which are not currently claimed but which may be claimed in the future, in particular if they are included within the scope of the independent claims.

Explanations relating to devices, apparatus, arrangements, systems, etc. according to embodiments of the invention may also apply to methods, processes, procedures, etc. according to embodiments of the invention and vice versa. Elements, process steps, etc. that are identical, have the same effect, correspond to one another in terms of their function, are structurally identical or have a comparable structure can be indicated with identical reference symbols.

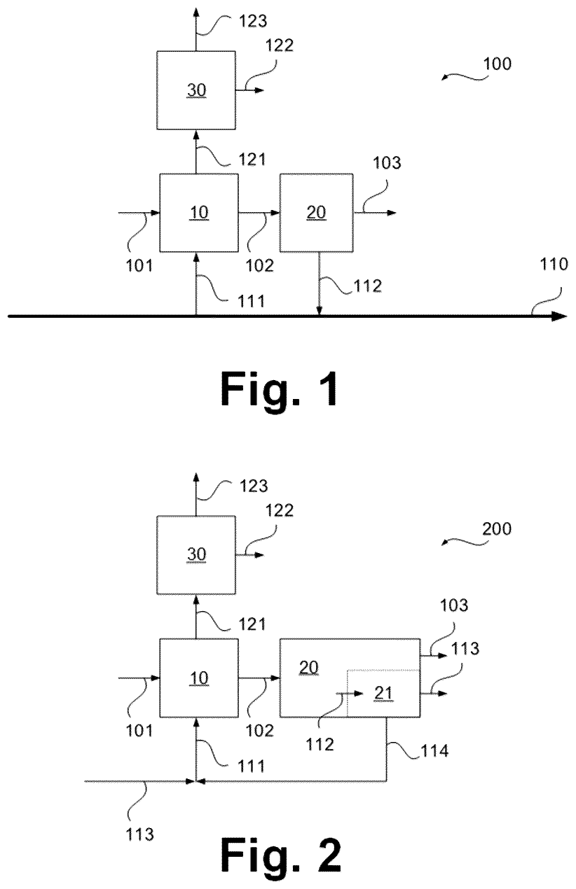

In FIG. 1, a process according to an embodiment of the invention is illustrated and designated overall by 100.

The process illustrated in FIG. 1 comprises one or more reaction steps 10, to which a reaction feed 101 is supplied, which is composed depending on the design of the reaction step(s) 10 and in particular contains one or more paraffins. The reaction feed 101 is reacted to obtain a product mixture 102, wherein one or more of the processes explained in detail above can be used.

The reaction step(s) 10 are carried out using heat of combustion provided by combustion of a methane-containing fuel gas 111, which can be taken from a pipeline 110 in the example of the process 100 illustrated in FIG. 1. During combustion, a flue gas 121 is formed which is subjected to carbon dioxide removal 30. Carbon dioxide separated here is designated 122, and flue gas which is depleted in carbon dioxide is designated 123.

The product mixture 102, which contains one or more olefins, hydrogen and methane, is now subjected to one or more separation steps 20, wherein one or more product fractions 103, which substantially contain the one or more olefins, and a tail gas fraction 112, which is substantially free of the one or more olefins and contains at least a portion of the hydrogen and methane from the product mixture, are formed.

Embodiments of the invention are characterized in that a mole ratio of hydrogen to methane in the tail gas fraction 112 is at least as high or higher than a mole ratio of hydrogen to methane in the product mixture 102, and that a mole ratio of hydrogen to methane in the fuel gas 111 is lower than that of the tail gas fraction 112.

The tail gas 112 is permanently discharged from the process 100. More specifically, the fuel gas 111 is removed from a gas supply line 110 and the tail gas 112 is fed back into the gas supply line 110.

In FIG. 2, a process according to an embodiment of the invention is illustrated and designated overall by 200.

As illustrated in FIG. 2, the tail gas 112 is subjected to a tail gas separation step 21 to obtain a first tail gas fraction 113 which is enriched in hydrogen and depleted in methane compared to the tail gas 112, and a second tail gas fraction 114 which is depleted in hydrogen and enriched in methane compared to the tail gas 112. The first tail gas fraction 113 or a part thereof may be permanently discharged from the process 200, but it may also be used for other purposes as previously explained. The use of the second tail gas fraction 114 was also explained.

For the production of pure hydrogen as a “low-carbon” energy carrier, a very simplified example is given below comparing an “on purpose” variant for the production of blue hydrogen using, for example, autothermal reforming (ATR) and carbon dioxide capture from the synthesis gas (Table 1), and a variant according to an embodiment of the invention using a cracker and carbon dioxide capture (Table 2).

TABLE 1

| | | |
|---|---|---|
| Hydrogen produced | MW | 100.000 |
| Loss in the ATR | MW H₂/MW CH₄ | 0.750 |
| Methane feed | MW | 133.333 |
| Carbon dioxide | (t/h)/MW CH₄ | 0.197 |
| Carbon dioxide | t/h | 26.311 |
| Carbon dioxide | (t/h)/MW H₂ | 0.263 |

TABLE 2

| | | |
|---|---|---|
| Hydrogen from cracker | MW | 100.000 |
| Reduced furnace efficiency | MW H₂/MW CH₄ | 0.979 |
| Methane replacement to the furnace | MW | 102.151 |
| Additional methane for carbon dioxide | MW | 18 |
| capture | | |
| Total feed of methane | MW | 120 |
| Carbon dioxide | (t/h)/MW CH₄ | 0.197 |
| Carbon dioxide | t/h | 23.710 |
| Total energy for amine scrubbing | GJ/t carbon dioxide | 2.73 |
| Carbon dioxide | (t/h)/MW H₂ | 0.237 |

The “on purpose” variant for the production of blue hydrogen according to Table 1 requires approximately 133 MW of methane (75% efficiency) for 100 MW of hydrogen, or in the best case 125 MW (80% efficiency). Corresponding amounts of carbon dioxide must be removed by carbon dioxide capture. The additional small energy gain in the form of export steam is not taken into account here.

The integrative variant according to an embodiment of the invention and Table 2 also causes additional energy consumption via carbon dioxide capture, but this is lower at approximately 120 MW of methane per 100 MW of hydrogen. This does not yet take into account that the process significantly simplifies the separation of carbon dioxide from the flue gas (due to the higher partial pressure). Also not taken into account was the energy required to separate methane and hydrogen, which, however, is fully electrifiable and would also be required for the variant shown in Table 1.

In summary, it can be concluded that the variant according to the invention requires at least 10% less thermal energy and produces 10% less carbon dioxide for the same production quantity of hydrogen and, in addition, no “on purpose” process (system for the production of blue hydrogen) is required. This is offset by the flue gas scrubbing for the steam cracker and the separation of methane and hydrogen (which, as mentioned, would also be required for the other system). However, this investment pays off both for the decarbonization of the olefin and the “low-carbon” energy stream. Another positive aspect is that the variant according to the invention causes fewer difficulties with regard to the formation of nitrogen oxides during combustion.

1. A process for producing one or more olefins, comprising:
reacting one or more paraffins to obtain a product mixture in one or more reaction steps, wherein;
the reaction step(s) are carried out using heat of combustion provided using a fuel gas, wherein a flue gas containing carbon dioxide is formed during the provision of the heat of combustion; and
the product mixture contains the one or more olefins, hydrogen and methane,
forming a tail gas using the product mixture or a part thereof in one or more separation steps, wherein the tail gas is substantially free of the one or more olefins and contains the hydrogen and the methane from the product mixture or a part thereof;
permanently discharging the tail gas from the process at a system boundary of a system used in the process; and
subjecting the flue gas or a part thereof to carbon dioxide capture;
wherein a mole ratio of hydrogen to methane in the tail gas is at least as high as in the product mixture, and in the fuel gas is lower than in the tail gas.

2. A process for producing one or more olefins, comprising:
reacting one or more paraffins to obtain a product mixture in one or more reaction steps, wherein:
the reaction step(s) are carried out using heat of combustion provided using a fuel gas, wherein a flue gas containing carbon dioxide is formed during the provision of the heat of combustion; and
the product mixture contains the one or more olefins, hydrogen and methane;
forming a tail gas using the product mixture or a part thereof in one or more separation steps, wherein the tail gas is substantially free of the one or more olefins and contains the hydrogen and the methane from the product mixture or a part thereof;
subjecting the flue gas to carbon dioxide capture:
subjecting the tail gas or a part thereof to one or more tail gas separation steps to obtain a first tail gas fraction enriched in hydrogen and depleted in methane compared to the tail gas and a second tail gas fraction depleted in hydrogen and enriched in methane compared to the tail gas;
permanently discharging the first tail gas fraction from the process at a system boundary of a system used in the process or using the first tail gas fraction to provide further heat of combustion;
wherein:
a mole ratio of hydrogen to methane in the tail gas is at least as high as in the product mixture, and in the fuel gas is lower than in the tail gas; and
the fuel gas comprises the second tail gas fraction or a part thereof.

3. The process according to claim 1, wherein the fuel gas or a part thereof is removed from a gas supply line and the tail gas is fed back into the gas supply line.

4. The process of claim 2, wherein the one or more tail gas separation steps comprise one or more membrane separation steps and/or one or more adsorptive separation steps.

5. The process according to claim 1, wherein during the carbon dioxide capture, more than 60% of the carbon dioxide contained in the flue gas or the part thereof subjected to the carbon dioxide capture is captured.

6. The process according to claim 1, wherein the carbon dioxide capture is carried out using one or more absorption liquids.

7. The process according to claim 1, wherein the fuel gas or a part thereof is combusted using an oxygen-enriched oxidizer gas.

8. The process according to claim 1, wherein the reaction step(s) is or are further carried out using electrically provided heat.

9. The process according to claim 1, wherein the fuel gas is a methane-rich gas or gas mixture.

10. (canceled)

11. (canceled)

12. (canceled)

13. The process according to claim 2, wherein during the carbon dioxide capture, more than 60% of the carbon dioxide contained in the flue gas or the part thereof subjected to the carbon dioxide capture is captured.

14. The process according to claim 2, wherein the carbon dioxide capture is carried out using one or more absorption liquids.

15. The process according to claim 2, wherein the fuel gas or a part thereof is combusted using an oxygen-enriched oxidizer gas.

16. The process according to claim 2, wherein the reaction step(s) is or are further carried out using electrically provided heat.

17. The process according to claim 2, wherein the fuel gas is a methane-rich gas or gas mixture.

18. The process according to claim 3, wherein during the carbon dioxide capture, more than 60% of the carbon dioxide contained in the flue gas or the part thereof subjected to the carbon dioxide capture is captured.

19. The process according to claim 4, wherein during the carbon dioxide capture, more than 60% of the carbon dioxide contained in the flue gas or the part thereof subjected to the carbon dioxide capture is captured.

20. The process according to claim 3, wherein the carbon dioxide capture is carried out using one or more absorption liquids.

21. The process according to claim 4, wherein the carbon dioxide capture is carried out using one or more absorption liquids.

22. The process of claim 1, wherein the one or more tail gas separation steps comprise one or more membrane separation steps and/or one or more adsorptive separation steps.
